(19) United States
(12) Patent Application Publication
(10) Pub. No.: US 2023/0285566 A1
(43) Pub. Date: Sep. 14, 2023

(54) ANTIBIOTIC THERAPEUTICS AND USES THEREOF

(71) Applicant: GENTAGEL LR LTD., Jerusalem (IL)

(72) Inventor: Abraham Jakob DOMB, Jerusalem (IL)

(21) Appl. No.: 18/040,436

(22) PCT Filed: Aug. 5, 2021
(86) PCT No.: PCT/IL2021/050954

§ 371 Date: Feb. 3, 2023

Related U.S. Application Data
(60) Provisional application No. 63/062,563, filed on Aug. 7, 2020.

(51) Int. Cl. A61K 47/34; A61K 31/351; A61P 31/04

(52) U.S. Cl. CPC A61K 47/34; A61K 31/351; A61P 31/04

(57) ABSTRACT

The invention concerns an antimicrobial formulation comprising at least one antibiotic agent and a polyanhydride carrier.

### FIELD OF THE INVENTION

The present invention relates to compositions of antibiotic therapeutics and uses thereof.

### BACKGROUND OF THE INVENTION

Biodegradable drug delivery systems are advantageous because they obviate the need for additional medical intervention for removal of non-degradable drug depleted devices. These polymers and their degradation components must possess several attributes including compatibility with biological tissues, negligible toxicity and easy elimination from the body. Biodegradable polymers are generally hydrophobic thereby maintaining their integrity in physiological environments after administration.

Biodegradable systems containing antibiotics such as gentamicin have been developed. However, they often provide inconstant release of the antibiotics. In addition, some of these systems have been reported to impart localized hypersensitivity reactions.

Previous in vitro and in vivo studies have shown that poly(ester-anhydrides) formed from ricinoleic and sebacic acids can serve as convenient and safe biodegradable polymers for the local administration of drugs. These copolymers [1] were also evaluated specifically for gentamicin administration in the treatment of osteomyelitis, showing good tolerability, favorable local release dynamics and no signs of inflammatory adverse reactions.

WO 2016/097848 [2] discloses a copolymer characterized by alternating or semi-alternating ester and anhydride bonds, methods for its production and use thereof, particularly as a carrier for drug delivery. The copolymer is characterized by reproducible product specifications including controlled viscosity and molecular weight and is shown to be stable for months at room temperatures.

WO 2018/178963 [3] discloses a depot system containing at least one antibiotic and a biodegradable poly(ester-anhydride) to provide prolonged local release of the antibiotic at the site of injection while maintaining the systemic antibiotic levels at sub-therapeutic concentrations.

While the biodegradable systems for local delivery of antibiotics overcome many of the shortcomings of prior non-biodegradable local treatments, they may not be sufficient to completely eradicate the bacteria involved in, e.g., formation of bone and teeth-related infections. Accordingly, additional advancements in therapeutic modalities are in need.

Polyanhydrides have been investigated as carriers for the controlled delivery of several drugs due to their surface eroding properties. Polyanhydrides have inherent high reactivity toward water, which prompts rapid hydrolytic degradation. Due to the high rate of hydrolysis, polyanhydrides endure surface erosion rather than bulk degradation. Polyanhydride based particles have been widely studied in many formulations for effective drug delivery. Nevertheless, the number of polyanhydride products existing in the market is fewer compared to polyester. Even though polyanhydrides are easy and inexpensive to synthesize and scale up, they exhibit a short shelf-life. Polyanhydrides are prone to hydrolytic degradation and depolymerization via anhydride interchange during storage, and may therefore be produced along with decomposition products. Hence, polyanhydrides need to be kept at freezing storage conditions that restrict their usage in drug delivery products. Accordingly, the usability of polyanhydride products in the medical fields (e.g. carriers of drugs) is less attractive. One such example is the poly(ester-anhydride) based on the ricinoleic acid and sebacic acid reported in [4-6].

### REFERENCES

[1] Brin et al., 2009, J Biomater Sci Polym Ed, 20, 1081-1090; Krasko et al., 2007 J. Control Release, 117, 90-96;
[2] WO 2016/097848;
[3] WO 2018/178963;
[4] U.S. Pat. No. 10,774,176;
[5] US 2020/0101163;
[6] Domb et al., 2017, J of Controlled Release, 257, 156-162.

### SUMMARY OF THE INVENTION

This invention disclosed herein concerns a unique biodegradable and biocompatible polymer-based composition for delivery of antibiotics of unlimited varieties. The formulations of the invention may be injected or inserted into a tissue for achieving maximum effect, or may even be applied topically for achieving local non-systemic effect. The delivery system provides a high local concentration of an antibiotic drug, thus achieving not only treatment of an existing condition or infection, but also preventing re-forming of the infection, over extended periods of time.

Formulations of the invention are generally based on a polyanhydride exhibiting improved properties to those previously disclosed in the art. The polyanhydride is a narrow-polydispersed polymer constructed of sebacic acid (SA) and ricinoleic acid (RA), prepared by melt condensation of SA and RA with a mole equivalent or less of acetic anhydride per carboxylic acid group, and in the absence of a solvent. The polyanhydride is of the form —(SA-RA)n-, wherein SA is sebacic acid and RA is ricinoleic acid, and wherein n is an integer between 10 and 100. This polyanhydride is referred to herein as the polymer of the invention or the carrier of the invention.

The absence of a solvent and the sequential addition of the various precursors allows for producing a final product that is well characterized and reproducible to meet regulatory requirements of the highest standards and which exhibits narrow polydispersity. The term “narrow polydispersity” or any lingual variation thereof, when made in reference to a polymer of the invention defines a collection of materials having substantially identical compositions (type of repeating groups and manner of repetition) and molecular weights. The narrow polydispersity of a polymer of the invention, defined by the ratio Mw/Mn (wherein Mw is the weight-average molecular weight and Mn is the number-average molecular weight) is below 2.5 or below 2. Putting it differently, the narrow disperse or narrow polydisperse polymer of the invention has a polydispersity value of no more than 2.5 or 2 (or a value between 2.5 and 1, or between 2 and 1).

Polymers of the invention also exhibit high reproducibility, namely a reproducibility in polymer molecular weight that is no more than 30% deviation from polymer average molecular weight.

The term “in absence of a solvent” herein refers to the property of the process of the invention as having no or a minute amount of solvent(s) that may be derived from impurities present with the precursor materials. Such impurities will not exceed 0.001%, 0.005%, 0.01%, 0.05% or 0.1% (w/w) of the total weight of the reaction materials used.

The polymer of the invention is prepared by a process comprising:
reacting sebacic acid (SA) and ricinoleic acid (RA) under conditions permitting esterification of the SA (to obtain a mono ester of SA or a di-ester thereof or a mixture thereof); and
transforming the (mono or di- or mixture thereof) esterified SA into the narrow-polydisperse polyanhydride.

The process of the invention permits for direct condensation in bulk (in the melt), without a pre-reaction to form a polymer or an oligomer of any of the material precursors used. In an exemplary process, sebacic acid (SA) (a dicarboxylic acid) was reacted with ricinoleic acid (RA) (a hydroxyl-alkanoic acid) at a 30:70 w/w ratio to form a mixture of SA-RA dimers and RA-SA-RA trimers with minimal or no RA or RA-RA ester molecules in the reaction product. The SA-RA and RA-SA-RA mixture (free of the precursor molecules and of the RA-RA molecules) is thereafter treated with no more than one molar equivalent of acetic anhydride per free carboxylic acid group (being typically 2 free carboxylic acid groups and thus no more than 2 molar equivalents) to acetylate the free ester and thereafter polymerize the acetylated segments into the narrow-dispersed polyanhydride having the repeating . . . RA-SA-RA-SA . . . sequence. The process is depicted in FIG. 1.

Mixture of dimers and trimers of SA and RA can be used to form a heterogeneous polymer consisting anhydride bonds and ester bonds between SA and RA with minimal ester bonds between two RA units. On the other hand, formation of anhydride diads of the SA monomers along the polymer chain, may limit the storage stability of the polymer. Thus, in a process of the invention, the molar ratio between a SA and RA is typically equivalent or in favor of RA. In other words, the amount of the RA is preferably equal to or double (1:1 to 1:2 molar equivalent) that of SA. In some embodiments, the weight ratio SA:RA is 1:1, 1:1.1, 1:1.2, 1:1.3, 1:1.4, 1:1.5, 1:1.6, 1:1.7, 1:1.8, 1:1.9, 1:2, respectively.

In some embodiments, the molar ratio between the SA:RA ranges between 1:1 and 1:2, respectively to avoid ester bond formation between RA units, so that the polymer comprises anhydride bonds and ester bonds only between SA and RA.

In some embodiments, the weight ratio is 30:70, 35:65 or 25:75 for SA and RA building blocks, respectively.

An excess amount of the RA permits mono- and diesterification of the SA (with some amount of a mono esterified form), and avoids formation of ester dimers of the RA. The SA-RA and SA-RA-SA mixture (herein a “dimer-trimer mixture”) is obtained by heating a mixture of SA and RA, in the indicated ratios, at a temperature above 80° C. In some embodiments, the temperature is between 80 and 200, between 100 and 190, between 100 and 180, between 100 and 170, between 100 and 160, between 100 and 150, between 100 and 140, between 100 and 130, or between 100 and 120° C.

The condensation of the two components involves direct ester condensation to provide the dimer-trimer dicarboxylic acid oligomer mixture. The dimer-trimers oligomers are polymerized into a polyanhydride by activation of the carboxylic acid ends with acetic anhydride. The amount of the acetic anhydride used is not greater than one molar equivalent of acetic anhydride per every free carboxylic acid group in the oligomers. The dimer SA-RA has two free carboxylic acid groups. Similarly, the trimer SA-RA-SA has 2 free carboxylic acid groups. Thus, no more than 2 molar equivalents of acetic anhydride may be used. In some embodiments, the amount of acetic anhydride is 2, 1.9, 1.8, 1.7, 1.6, 1.5, 1.4 or 1.3 molar equivalents.

In some embodiments, the acetylation step may be carried out at a temperature above 40° C. In some embodiments, the acetylation temperature is between 40° C. and the boiling point of acetic anhydride. In some embodiments, the acetylation temperature is between 40 and 90, between 40 and 100, between 40 and 110, between 80 and the boiling point of the acylation anhydride. The temperature used for the acylation-activation of the oligomers is a function of time, the longer the reaction time, the lower the temperature to be applied. It is possible to react the diacid oligomers with acetic anhydride under pressure to expedite the reaction or perform the reaction under microwave heating. These methods require tuning the reaction conditions so that the oligomers are acetylated and not deteriorated. Moreover, other acetylation methods may apply, including reaction with acetyl chloride with an acid scavenger.

The temperature may be increased following acetylation to condense the acetylated precursors to form the aforementioned dimer/trimer mixture.

The transforming into the narrow-polydispersed polymer of the invention is achieved by polymerization. Polymerization of the dimer-trimer mixture into a polymer of the invention may be achieved by heating the acetylated dimers and trimers under low pressure and elevated temperatures. In some embodiments, polymerization is achievable in vaccuo and heating. The thermal conditions may involve heating the acetylated dimer-trimer mixture to a temperature between 100 and 200, between 100 and 190, between 100 and 180, between 130 and 170, between 130 and 160, between 130 and 150, or between 130 and 140° C. In some embodiments, the temperature is between 120 and 170 or between 130 and 160° C. The reaction time is an important parameter, as the higher the reaction temperature, the shorter is the reaction time. There is a minimum time required for forming the oligomers and polymers; longer reaction time has no or little effect on the oligomer composition or polymer molecular weight. The reaction time is dependent on the batch size and the reaction conditions, including the mixing method and rate and vacuum profile applied.

In some embodiments, polymerization is achievable at high thermal conditions, as specified, under vacuum.

In some embodiments, the process comprises:
reacting SA and RA at a temperature between 80 and 200° C. to obtain a mixture of a mono ester (SA-RA) and a diester (SA-RA-SA) of SA; and
reacting the mixture with acetic anhydride under conditions permitting polymerization of the mono ester and diester into the polyanhydride.

In some embodiments, the process comprises:
reacting SA and RA at a temperature between 80 and 200° C. to obtain a mixture of a mono ester (SA-RA) and a diester (SA-RA-SA) of SA; and
reacting the mixture with acetic anhydride to acetylate the mixture of monoester and diester; and
thermally treating the acetylated mixture under conditions permitting polymerization into the polyanhydride.

In some embodiments, the process comprises:
reacting SA and RA in the presence of acetic anhydride at a temperature between 80 and 200° C. to obtain a mixture of a mono ester and a diester of SA, as herein; and
thermally treating the acetylated mixture in vaccuo at a temperature between 100 and 200° C., permitting polymerization to afford the polyanhydride.

The polymer of the invention is thus a narrow-polydisperse polyanhydride of the formula —(SA-RA)n-, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2, prepared by a process as disclosed above, where the mixture or dimer and trimer dicarboxylic acids are linked to form a chain by anhydride bonds. Processes of the invention exclude such processes which produce polydisperse polyanhydrides. Processes of the invention are free of steps forming or utilizing a polymer or oligomer derived from (consisting) SA or derived from (consisting) RA. One such process is excluded from the scope of the present invention is a process utilizing SA and RA and disclosed in publications [4-6]. The polymer of the invention is subject of co-pending U.S. patent application No. 63/062,563 and any co-pending applications claiming priority therefrom, each of which herein incorporated by reference.

Thus, the carrier in all its embodiments is prepared by methods or processes as herein, wherein the method or process or preparation does not comprise use of poly sebacic acid.

The highly reproducible batch-to-batch polymer molecular weight provide improved reproducible viscosity allowing predictable injectability, highly reproducible compositions and drug release profiles, alongside a polymer degradation rate that is predictable, manageable, with a narrow standard deviation, and a high purity (minimal or no reactant impurities of acetic anhydride and anhydride molecules), the polymers of the invention are superior to those discussed in the art. Accordingly, the usability of polyanhydrides of the invention in the medical fields, e.g. as drug carriers, opens the door for a new generation of drug carriers.

Thus, in a first aspect there is provided an antibiotic or antimicrobial formulation comprising a polymer of the invention (as defined or as prepared) and at least one antibiotic agent.

More specifically, formulations of the invention comprise at least one antibiotic agent and a carrier in a form of a polyanhydride composed of sebacic acid (SA) and ricinoleic acid (RA), the carrier having a Mw/Mn value between 1 and 2.5. The carrier is a polyanhydride of the formula —(SA-RA)n-, wherein n is an integer between 10 and 100. As noted herein, the polyanhydride is prepared by: a. melt condensation of SA and RA to form dicarboxylic acid oligomers; b. oligomer activation with acetic anhydride; c. melt polycondensation to form a polyanhydride. Oligomer activation is achievable in the presence of a mole equivalent or less of acetic anhydride per carboxylic acid group, in the absence of a solvent.

As used herein, the antibiotic or antimicrobial; “formulation” is a pharmaceutical grade formulation or composition comprising at least one antibiotic agent and a carrier that comprises or consists a polymer of the invention. Where properties of a formulation of the invention are to be modified, in some embodiments, the carrier utilized may comprise in addition to a polymer of the invention also other acceptable carriers such as, for example, vehicles, adjuvants, excipients, or diluents. The choice of using a further carrier in addition to a polymer of the invention will be determined in part by the particular antibiotic agent, as well as by the particular method used to administer the formulation and by the particular form of the formulation.

In some embodiments, the antibiotic/antimicrobial formulation comprises an antibiotic agent and a carrier in a form of a polyanhydride of the formula —(SA-RA)n-, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

In some embodiments, the polyanhydride is prepared by melt condensation of SA and RA with a mole equivalent or less of acetic anhydride per carboxylic acid group, in the absence of a solvent. In other words, the polyanhydride is not prepared by processes involving use of a solvent or polymerization of RA or SA alone.

The invention also provides use of a carrier in a form of a polyanhydride of the formula —(SA-RA)n-, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2, for preparing an antibiotic formulation comprising at least one antibiotic agent.

Further, an antibiotic agent is provided for the preparation of an antibiotic formulation comprising the antibiotic agent and a carrier in a form of a polyanhydride of the formula —(SA-RA)n-, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

Formulations of the invention comprising an antibiotic agent and a polymer of the invention may be formed by a variety of ways. In some cases, formulations are formed by mixing a polymer of the invention, as defined, with the at least one antibiotic agent. In such cases, a measurable dosage amount of the antibiotic agent is mixed with an appropriate amount of the polymer to obtain a homogenous formulation. In other cases, formulations are formed by mixing the antibiotic agent with the polymer precursors during preparation of the polymer.

Generally speaking, formulations of the invention may be configured as controlled release formulations. The term “controlled delivery” is used herein in its broadest sense to denote a formulation whereby discharge of the antibiotic agent from the formulation and permeation of agent through tissues, its accessibility and bioavailability in tissues and blood circulation, and/or targeting to the specific tissues of action are modulated to achieve specific effects over time. It encompasses immediate, prolonged, and sustained delivery of the antibiotic agent, drug protection against degradation, preferential metabolism, clearance or delivery to specific tissues. Controlled release of the antibiotic agent included in a formulation of the invention can be obtained by several means, as known in the art.

Typically, formulations of the invention are configured as prolonged delivery or sustained delivery formulations.

The term “prolonged delivery” implies a delayed permeation and/or release of the antibiotic agent from the formulation and into the tissue. In other words, in a prolonged delivery, the agent can be detected or measured in the tissue or circulation after a lag period, and in this case, after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering. The prolonged delivery also applies to target organs and tissues with additional lag of at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering.

The term “sustained delivery” implies a profile of continued released and/or permeation of the agent from the formulation and into the tissue or circulation, or in other words, that the relates and/or permeation of the agent from the formulation and into the tissue or circulation reaches a plateau or a steady state after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering, and that the plateau or the steady state persists for at least about 1 h, 2 h, 3 h, 4 h, 5 h, 6 h, 7 h, 8 h, 9 h, 10 h, 11 h, 12 h, 13 h, 14 h, 15 h, 16 h, 17 h, 17 h, 18 h, 19 h, 20 h or more after.

The “antibiotic” agent is a drug intended for use by humans or animals to inhibit or destroy or prevent infection by a microorganism or treat or prevent development of a disease mediated or caused by a bacterium. The term does not encompass antibiotic materials having chemotherapeutic activity. The antibiotic agent used in accordance with the invention, is any such agent known to have antibacterial or antimicrobial activity. Putting it differently, the antibiotic is any such agent administered to a subject to achieve treatment or prophylaxis of an infection caused by bacteria or some parasites. In some embodiments, the bacteria are cocci bacteria, bacillus bacteria, rickettsia bacteria, mycoplasma bacteria, and others.

In some embodiments, the bacteria are selected amongst Gram-positive and Gram-negative bacteria.

In some embodiments, the antibiotic agent is selected to treat or prevent an infection caused by Gram-positive bacteria such as Streptococcus, Staphylococcus and Clostridium botulinum. In some embodiments, the antibiotic agent is selected to treat or prevent an infection caused by Gram-negative bacteria such as Cholera, Gonorrhea, Escherichia coli (E. coli), Pseudomonas aeruginosa and Acinetobacter baumannii.

In some embodiments, the antibiotic agent is selected to treat or prevent an infection caused by a bacterium selected from Aerococcus urinae, Chlamydia trachomatis, Enterococcus faecalis, Fusobacterium necrophorum, Fusobacterium nucleatum, Moraxella catarrhalis, Neisseria gonorrhoeae, Neisseria meningitides, Pediococcus damnosus, Staphylococcus aureus, Staphylococcus haemolyticus, Staphylococcus saprophyticus, Streptococcus agalactiae, Streptococcus bovis, Streptococcus pneumoniae, Streptococcus pyogenes, Aeromonas hydrophila, Arcanobacterium bemolyticum, Bacillus anthracis, Capnocytophaga canimorsus, Chlamydophila pneumoniae, Chlamydophila psittaci, Clostridium botulinum, Clostridium difficile, Clostridium tetani, Corynebacterium diphtheriae, Corynebacterium jeikeium, Escherichia coli, Klebsiella aerogenes, Legionella pneumophila, Listeria monocytogenes, Mycobacterium leprae, Mycobacterium tuberculosis, Plesiomonas shigelloides, Prevotella intermedia, Porphyromonas gingivalis, Propionibacterium acidipropionici, Providencia stuartii, Salmonella typhimurium, Serratia marcescens, Vibrio cholerae, Vibrio vulificans, Brevibacterium linens, Rickettsia akari, Rickettsia conorii, Rickettsia felis, Rickettsia prowazekii, Rickettsia rickettsii, Rickettsia typhi, Borrelia afzelii, Borrelia burgdorferi, Borrelia hermsii, Campylobacter coli, Helicobacter hepaticus, Helicobacter pylori, Leptospira interrogans, Spirillum minus, Treponema pallidum, Treponema carateum, Treponema denticola, Mycoplasma fermentans, Mycoplasma gallisepticum, Mycoplasma genitalium, Mycoplasma haemofelis, Mycoplasma hominis, Mycoplasma hyopneumoniae, Mycoplasma incognitus, Mycoplasma penetrans, Mycoplasma pneumoniae, and others.

In some embodiments, the antibiotic agent is selected based on its ability to treat or prevent a disease or a condition mediated or caused by a bacterium. Generally speaking, a bacterium can cause a disease by a variety of mechanisms: (1) by secreting or excreting toxins, as in botulism, (2) by producing toxins internally, which are released when the bacteria disintegrate, as in typhoid, (3) or by inducing sensitivity to their antigenic properties, as in tuberculosis. Other mechanisms may be involved as well. Thus, the disease or the condition may be any one or more of botulism, typhoid, tuberculosis, cholera, diphtheria, bacterial meningitis, tetanus, Lyme disease, gonorrhea, and syphilis.

The antibiotic agent may be selected amongst Penicillins, Tetracyclines, Cephalosporins, Quinolones, Lincomycins, Macrolides, Sulfonamides, Glycopeptides, Aminoglycosides, and Carbapenems.

In some embodiments, the antibiotic agent is amoxicillin, amoxicillin, ampicillin, dicloxacillin, oxacillin, penicillin V potassium, demeclocycline, doxycycline, eravacycline, minocycline, omadacycline, tetracycline, cefaclor, cefdinir, cefotaxime, ceftazidime, ceftriaxone, cefuroxime, ciprofloxacin, levofloxacin, moxifloxacin, clindamycin, lincomycin, azithromycin, clarithromycin, erythromycin, dalbavancin, oritavancin, telavancin, vancomycin, gentamycin, tobramycin, amikacin, imipenem, cilastatin, meropenem, doripenem, ertapenem, and others, or pharmaceutically acceptable salts thereof.

In some embodiments, the antibiotic agent is at least one of aztreonam, cefuroxime, cephalexin, clindamycin, vancomycin, ceftazidime, cefazolin, ceftriaxone, cephalosporin, piperacillin, tazobactam, tobramycin, levofloxacin, amoxicillin, clavulanic acid, and gentamicin, or pharmaceutically acceptable salts thereof.

In some embodiments, the antibiotic agent is cefuroxime.

In some embodiments, the antibiotic agent is an aminoglycoside. In some embodiments, the aminoglycoside antibiotic is at least one of kanamycin A, amikacin, tobramycin, dibekacin, gentamicin, sisomicin, netilmicin, neomycins B, C or E, and streptomycin, or pharmaceutically acceptable salts thereof.

In some embodiments, the aminoglycoside antibiotic is gentamicin or a pharmaceutically acceptable salt thereof (e.g. gentamicin sulfate).

In some embodiments, the antibiotic agent is at least one of apramycin, arbekacin, astromicin, bekanamycin, dihydrostreptomycin, elsamitrucin, fosfomycin/tobramycin, G418, hygromycin B, isepamicin, kasugamycin, legonmycin, lividomycin, micronomicin, neamine, nourseothricin, paromomycin, plazomicin, ribostamycin, streptoduocin, totomycin, and verdamicin, or pharmaceutically acceptable salts thereof.

In some embodiments, the antibiotic agent is at least one of ampicillin, norfloxacin, sulfamethoxazole, flumequine, and amphotericin B, or pharmaceutically acceptable salts thereof.

Further provided by the invention are methods of treatment or prevention utilizing formulations of the invention.

In one aspect, there is provided a method for treating or delaying or preventing progression of an infectious disease or disorder, e.g., mediated by at least one bacterium, the method comprising administering to a subject (human or non-human) an effective amount of an antibiotic agent in a formulation of the invention, as described herein.

The term “treatment” as used herein refers to the administering of a therapeutic amount of the formulation of the present invention which is effective to ameliorate undesired symptoms associated with a disease, e.g., an infectious disease, to prevent the manifestation of such symptoms before they occur, to slow down the progression of the disease (also referred to herein as “delaying the progression”), slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

The term “effective amount” as used herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The antibiotic agent may be present in formulations of the invention in an amount or dose, which amount will depend on a variety of considerations known to those versed in drug formulation. Without wishing to be bound by any dose amounts, typically the antibiotic agent may be present in an amount between 0.1 and 75% w/w, depending on the potency of the drug, the volume of formulation configured for, e.g., injection or topical, and the desired release profile. The hydrophobic nature of the polymer of the invention may protect, in part, the incorporated drug from being deteriorated due to light interaction, oxidation or hydrolysis during storage and in patient. The pasty polymer can be injected or spread on a diseased surface such as the lungs, colon and other tissues employing administration methodologies known in the art.

Formulations of the invention may be delivered by a variety of ways. In some embodiments, an effective amount of the antibiotic agent may be administered topically, orally or by injection. In some embodiments, the administrated is by one or more of the following routes oral, topical, transmucosal, transnasal, intestinal, parenteral, intramuscular, subcutaneous, intramedullary injections, intrathecal, direct intraventricular, intravenous, intraperitoneal, intranasal, or intraocular injections.

In some embodiments, the formulation is administered by injection.

In some embodiments, the formulation may be administered via use of a tablet, a pill, a capsule, pellets, granules, a powder, a lozenge, a sachet, a cachet, an elixir, a suspension, a dispersion, an emulsion, a solution, a syrup, an aerosol, a gel, an ointment, a lotion, a cream, and a suppository.

To achieve systemic administration, the formulation may be administered via oral, rectal, transdermal, parenteral (subcutaneous, intraperitoneal, intravenous, intra-arterial, transdermal and intramuscular), topical, intranasal, or via a suppository administration.

In some embodiments, the administration is a local administration to a site or in proximity or vicinity of a site of a diseased tissue or organ. The local administration may be topically or by injection.

As used herein, the term “local” as well as the terms “proximity” or “vicinity” with reference to a site of injection or delivery or site of local administration, refer to a radius of about 0 to about 10 cm from the site of diseased tissue or organ.

Methods of use and uses according to the invention utilize a carrier of the invention in a form of a polyanhydride of the formula —(SA-RA)n-, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

In some embodiments, the carrier is prepared by any of the processes disclosed herein.

In some embodiments, formulations used in accordance with the invention comprise the antibiotic agent, as defined, and a carrier, as defined, wherein the carrier is prepared by a process comprising melt polycondensation of RA and SA in presence of an amount of acetic anhydride not exceeding a mole equivalent thereof per each free carboxylic acid group and in absence of a solvent.

### BRIEF DESCRIPTION OF THE DRAWINGS

The invention may be more clearly understood upon reading of the following detailed description of non-limiting exemplary embodiments thereof, with reference to the following drawings, in which:

FIG. 1 is a synthetic scheme of a polyanhydride carrier of the present invention.

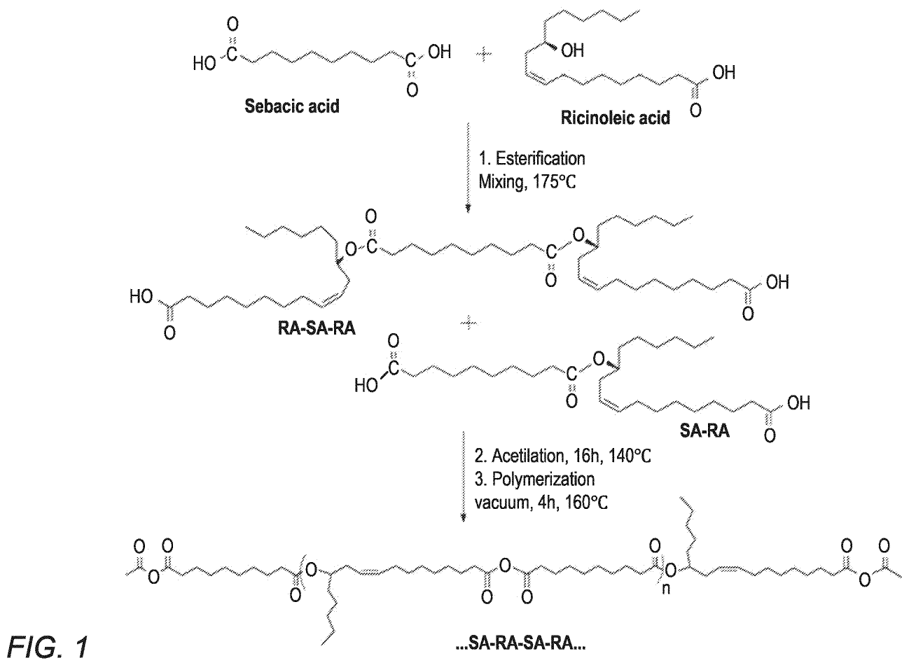

### DETAILED DESCRIPTION OF THE INVENTION

### Example 1: Controlled Synthesis of Oligomers of Different Type of Dicarboxylic Acid and Hydroxy Acids Forming a Carrier According to the Invention

Aim: development of an alternative method to synthesis of oligomers of different type of dicarboxylic acid and hydroxy acids.
Materials: Suberic acid (SUA) and dodecanedioic acid (DDDA) were used as received. Ricinoleic acid (RA) was prepared from the hydrolysis of castor oil as described in the synthesis part.

### Spectral Analysis

¹H and ¹³C NMR spectra were obtained on a Varian 300 MHz NMR spectrometer using CDCl₃ as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts).
Preparation of ricinoleic acid from castor oil: In a 1000 mL round bottom flask, 48 g of KOH was dissolved in 400 mL of ethanol by heating (65° C.). Then, 200 g of castor oil was added to it and mixed them properly. The mixture was then refluxed for 2 hr at 140° C. with continuous staring. After the reflux, the solvent was evaporated by evaporator. Then 200 mL of double distilled water, 150 mL diisopropyl ether, and 150 mL H₃PO₄ were added and the total mixture was transferred to a separating funnel. It was then repeatedly washed with double distilled water (3-5 times, 200 mL each time) until the pH of the aqueous phase ˜4. Then the organic phase was collected through sodium phosphate and evaporated to dryness to obtain pure 185 g of Ricinoleic acid (yield 92.5%), confirmed by ¹H NMR.
Synthesis of SUA-RA and DDDA-RA oligomers: SUA-RA and DDDA-RA oligomers were synthesized by esterification reaction of suberic acid and dodecanedioic acid with ricinoleic acid at 170° C. In a round bottom flask, 15 g of SUA, 15 g of RA and catalytic amount (1%) of phosphoric acid were taken and heated to 170° C. for 5 hours under nitrogen. Then another 15 g of RA was added to the round bottom flask and continued to heat for another 4 hours under nitrogen swift. Finally another 5 g of RA was added and again continued to heat over night with mixing under vacuum to yield SUA-RA oligomer with 30:70 ratios of SUA and RA which was characterized by ¹H NMR. DDDA-RA oligomer with 30:70 ratios of DDDA and RA was synthesized following the same procedure and was also characterized by ¹H NMR.
Discussion of the results: Two different oligomers are synthesized using two different dicarboxylic acid and hydroxy acids. RA is esterified with SUA or DDDA under melt and vacuum condition where H₃PO₄ is used as catalyst. Under this reaction condition 100% of the RA is consumed in the esterification reaction with SUA or DDDA which is confirmed from the ¹H NMR as the signal at 3.6 ppm for the alcoholic proton is gone astray after the final step of esterification. Furthermore, self-condensation of RA in this protocol (via step by step addition of RA to SUA or DDDA) is also avoided; evidence form ¹H NMR, as there is no signal at 4.1 ppm. Hence this process gives a well-defined SUA-RA or DDDA-RA oligomers without any residual or self-condensed RA.

### Example 2: Synthesis of Poly(Ester-Anhydride) Approaching from an Alternative Method

The objective is the development an alternative method to synthesis of biodegradable copolymer of poly(ester-anhydride). Here the focus is on two features:
1) Use of sebacic acid (SA) and ricinoleic acid (RA) or 12-hydroxystearic acid (HSA) to prepare SA-RA or SA-HSA oligomers by direct condensation.
2) Use of fewer amounts (1:1 equivalent or less) of acetic anhydride to activate the oligomers for polymerization.
3) Control the molecular weight of poly(ester-anhydride) depending upon amount the acetic anhydride used for the pre-polymerization step.
   Materials: Sebacic acid (SA, 99% pure; Aldrich, USA), 12-hydroxystearic acid (HSA) and acetic anhydride (Merck, Germany) were used as received. Ricinoleic acid (RA) was prepared from the hydrolysis of castor oil as described in the synthesis part.
   Spectral analysis: ¹H and ¹³C NMR spectra were obtained on a Varian 300 MHz NMR spectrometer using CDCl₃ as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts).
   Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, Calif.) injection valve with a 20 μL-loop. The samples were eluted with CHCl₃ (HPLC grade) through linear Styragel HR5 column (Waters) at a flowrate of 1 mL/min. The molecular weights were determined relative to polystyrene standards.
   Synthesis and Characterization: SA-RA oligomers: SA-RA oligomers were synthesized by heating ricinoleic acid and sebacic acid at 175° C. In a round bottom flask, 30 g of SA, 30 g of RA and catalytic amount (0.1%) of phosphoric acid were taken and heated to 170° C. for 5 hours under nitrogen. Then another 30 g of RA was added to the round bottom flask and continued to heat for another 4 hours under nitrogen swift. Finally, another 10 g of RA was added and again continued to heat over night with mixing under vacuum to yield SA-RA oligomer with 30:70 ratios of SA and RA which was characterized by ¹H NMR and FTIR. The SA-RA oligomers of different ratios were also prepared by the same process and characterized by ¹H NMR. The details are given in the Table 1 below.

TABLE 1
SA-RA oligomers

| | | RA | | |
|---|---|---|---|---|
| | | 1^{st} Step, | 2^{nd} Step | 3^{rd} Step |
| SA-RA | | 170° C., | 170 ° C., | 170° C., |
| ratio | SA | 5 hrs, N₂ | 4 hrs, N₂ | Overnight, Vacuum |
| 20:80 | 10 g | 17.5 g | 17.5 g | 5 g |
| 25:75 | 12.5 g | 16.25 g | 16.25 g | 5 g |
| 35:65 | 17.5 g | 13.75 g | 13.75 g | 5 g |

### SA-HAS Oligomers

SA-HSA oligomers were also synthesized by heating 12-hydroxystearic acid and sebacic acid at 175° C. In a round bottom flask, 15 g of SA, 15 g of HSA and catalytic amount (0.1%) of phosphoric acid were taken and heated to 170° C. for 5 hours under nitrogen. Then another 15 g of HSA was added to the round bottom flask and continued to heat for another 4 hours under nitrogen swift. Finally, another 5 g of HSA was added and again continued to heat over night with mixing under vacuum to yield SA-HSA oligomer with 30:70 ratios of SA and HSA which was characterized by ¹H NMR and FTIR. The SA-HSA oligomers of 20:80 ratios were also prepared by the same process. The details are given in the Table 2 below.

TABLE 2
SA-RA oligomers

| | | HSA | | |
|---|---|---|---|---|
| | | 1^{st} Step | 2^{nd} Step | 3^{rd} Step |
| SA-has | | 170° C., | 170° C., | 170° C., |
| ratio | SA | 5 hrs, N₂ | 4 hrs, N₂ | Overnight, Vacuum |
| 20:80 | 10 g | 17.5 g | 17.5 g | 5 g |

### Poly(SA-RA)

In a typical synthesis, 10 g of 20:80, 25:75, 30:70, 35:65 ratio of SA-RA oligomers were melt individually at 140° C. under nitrogen atmosphere. Then 1:5 equivalent of acetic anhydride was added to the molten SA-RA oligomers and refluxed at 140° C. for 60 min. Excess acetic anhydride or acetic acid was evaporated. The residue was then subjected to melt condensation at 160° C. under 10 mbar for 4 hours. The SA-RA oligomer of 30:70 ratios was also polymerized under same procedure where different amount (1, 0.7, 0.5, 0.35, 0.25, 0.15 equivalent) of acetic anhydride was used (refluxed at 140° C., overnight) to use fewer amount of acetic anhydride and make a control over the molecular weight.

### Poly(SA-HSA)

Following the same procedure as poly(SA-RA), 10 g of 20:80 and 30:70 ratio of SA-HSA oligomers were melt individually at 140° C. under nitrogen atmosphere. Then 1:5 equivalent of acetic anhydride was added to both of the molten SA-HSA oligomers and refluxed at 140° C. for 60 min. Excess acetic anhydride or acetic acid was evaporated. The residue was then subjected to melt condensation at 160° C. under vacuum (˜10 m bar) for 4 h.

### Discussion of the Results:

Two kinds of poly(ester-anhydride) copolymers were synthesized through solvent free melt polycondensation process where directly sebacic acid is used to synthesis the SA-RA or SA-HSA oligomers instead of using poly(SA) as starting material. RA or HAS is esterified with SA under melt and vacuum condition where about 1% H₃PO₄ is used as catalyst. Under this reaction condition 100% of the RA or HSA is consumed in the esterification reaction with SA which is confirmed from the ¹H NMR as the signal at 3.6 ppm for the alcoholic proton is gone astray after the final step of esterification. Furthermore, self-condensation of RA or HSA in this protocol (via step by step addition of RA or HAS to SA) is also avoided; evidence form ¹H NMR, as there is no signal at 4.1 ppm. Hence this process gives a well-defined SA-RA or SA-HSA oligomers without any residual or self-condensed RA or HSA. The proton of the esterified polymer chemical shift observed at ˜4.8 ppm. Two protons adjacent to the ester bonds and anhydride bonds arise at 2.43 ppm and 2.33 ppm, respectively.

The molecular weight of the as-synthesized polymers is measured by GPC. The details of the molecular weight and disparity are given in the below Table 3 and control over molecular weight depending upon the acetic anhydride used.

TABLE 3
molecular weight and disparity of polymers of the invention

| | | Molecular | polydis- |
|---|---|---|---|
| Sl. | | weight | persity |
| No | polymer | (M_{w}) Daltons | (PD) |
| 1 | Poly(SA-RA) with 20:80 ratio, using 1:5 | 17091 | 3.01 |
| | w/w acetic anhydride | | |
| 2 | Poly(SA-RA) with 25:75 ratio, using 1:5 | 18793 | 3.07 |
| | w/w acetic anhydride | | |
| 3 | Poly(SA-RA) with 30:70 ratio, using 1:5 | 12335 | 2.85 |
| | w/w acetic anhydride | | |
| 4 | Poly(SA-RA) with 35:65 ratio, using 1:5 | 18558 | 3.02 |
| | w/w acetic anhydride | | |
| 7 | Poly(SA-RA) with 30:70 ratio, using 0.5 | 4841 | 1.72 |
| | equivalent acetic anhydride | | |
| 8 | Poly(SA-RA) with 30:70 ratio, using | 3296 | 1.51 |
| | 0.35 equivalent acetic anhydride | | |
| 9 | Poly(SA-RA) with 30:70 ratio, using | 2357 | 1.35 |
| | 0.25 equivalent acetic anhydride | | |
| 10 | Poly(SA-RA) with 30:70 ratio, using | 1856 | 1.24 |
| | 0.15 equivalent acetic anhydride | | |
| 11 | Poly(SA-HSA) with 20:80 | 15498 | 3.18 |
| | ratio, using 1:5 w/w acetic anhydride | | |
| 12 | Poly(SA-HSA) with 30:70 | 17630 | 3.33 |
| | ratio, using 1:5 w/w acetic anhydride | | |

### Example 3: Synthesis of Poly(SA-RA) with Reduced Reaction Time

Aim: The aim of the project is to monitor the synthesis process via ¹H NMR of biodegradable copolymer of poly(sebacic acid-ricinoleic acid) to reduce the reaction time.
Materials: Sebacic acid (SA, 99% pure; Aldrich, USA) was used as received. Ricinoleic acid (RA) was prepared from the hydrolysis of castor oil as described in the synthesis part. Spectral analysis: ¹H NMR spectra were obtained on a Varian 300 MHz NMR spectrometer using CDCl₃ as solvent. Fourier transform infrared (FTIR) spectroscopy was performed using a Smart iTR ATR sampling accessory for Nicolet iS10 spectrometer with a diamond crystal (Thermo Scientific, Massachusetts).
Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, Calif.) injection valve with a 20 μL-loop. The samples were eluted with CHCl₃ (HPLC grade) through

linear Styragel HR5 column (Waters) at a flowrate of 1 mL/min. The molecular weights were determined relative to polystyrene standards.

Synthesis of SA-RA oligomer: SA-RA oligomers were synthesized by heating ricinoleic acid and sebacic acid at 170° C. In a round bottom flask, 15 g of SA, 15 g of RA and catalytic amount (0.1%) of phosphoric acid were taken and heated to 170° C. for 2 hours under nitrogen. Then another 15 g of RA was added to the round bottom flask and continued to heat for another 2 hours under vacuum for 15 min followed by nitrogen swift. Finally, 5 g of RA was added and again continued to heat for another 8 hours under vacuum to yield SA-RA oligomer with 30:70 w/w ratio of SA and RA which was characterized by ¹H NMR.
poly(SA-RA): In a typical synthesis, 10 g of SA-RA oligomer with 30:70 ratios were melted at 140° C. under nitrogen atmosphere. Then 1 equivalent of acetic anhydride with respect to the acid in the oligomer was added to the molten SA-RA oligomer and refluxed at 140° C. for 2 hours. Excess acetic anhydride or acetic acid was evaporated. The residue was then subjected to melt condensation at 160° C. under vacuum (˜10 m bar) for 4 hours.

### Discussion of the Results:

RA is esterified with SA under melt and vacuum condition where H₃PO₄ is used as catalyst. Under this reaction condition 100% of the RA is consumed within 12 hours in the esterification reaction with SA. This is confirmed by ¹H NMR, thus, as the signal at 3.6 ppm for the alcoholic proton is gone astray after the final step of esterification. Furthermore, self-condensation of RA in this protocol (via step by step addition of RA to SA) is also avoided; evidence form ¹H NMR, as there is no signal at 4.1 ppm. Then the oligomer was polymerized by refluxing at 140° C. with 1 equivalent of acetic anhydride for 2 hours followed by heating at 160° C. under vacuum for 4 hours. The molecular weight of the polymer is measured by GPC and compared with the polymer that is synthesized from the same SA-RA oligomer with 30:70 ratios by refluxing at 140° C. with 1 equivalent of acetic anhydride for overnight followed by heating at 160° C. under vacuum for 4 hours. It is noticed that both the process gives almost same molecular weight of the polymers (˜11500 Daltons).

### Example 4: Gentamicin In Vitro Release from Different Polymer Batches

The objective of this study was to determine the difference between the gentamicin formulations prepared from poly(SA=RA)30:70 pasty polymers prepared by the method of this invention where the mole ratio of acetic anhydride to carboxylic acid of the RA:SA oligomers was 0.8 and polymers prepared where the ratio was 5. The release of gentamycin from 20% gentamycin sulfate loaded in poly(SA=RA)30:70 pasty polymer of different batches in aqueous media was studied. This release study was determined in acetate buffer pH4.5 that was determined useful for the release of amino-containing molecules such as gentamicin. For comparison, the release in phosphate buffer pH7.4 at 37° C. was determined.

Five polymer samples, prepared similarly by the procedure of this invention using a 0.80 mole ratio of acetic anhydride to carboxylic acid and polymerization time of 4 hours at 160° C., under a vacuum of 15 mm Hg, having a weight average molecular weight, Mw=9400+/−300 and polydispersity of 1.35 were used. For comparison, five polymer samples prepared by using 5 mole ratio of acetic anhydride to carboxylic acid under same polymerization conditions, having a weight average molecular weight, Mw=12000+/−4200 and polydispersity of 3.2. The intrinsic viscosity of the polymers of this invention was 0.15+/−0.1 while the polymers prepared by the old procedure showed intrinsic viscosity of 0.20+/−0.5.

These polymers were used for the preparation of 20% loaded gentamicin sulfate as follows: Gentamicin (GM) was first dried by heating at 120° C. for 1 hour and then allowed to cool to room temperature in vacuum. This dried GM was then incorporated in P(SA-RA) (30:70). The incorporation was done by mixing the dry GM powder (20% w/w) with the polymer by trituration until a homogeneous paste was formed. If the polymer was viscous, heating the polymer to 40° C. was applied. The formulations were loaded in 2 ml glass syringes and the injectability through a 23G needle was determined. The blank polymers were also loaded in syringes for the injectability test. The blank polymers and the gentamicin loaded polymer formulations prepared by this invention with a narrow molecular weight and polydispersity showed same injectability with smooth release of the polymer or formulation from the syringes and needle with same force applied on the plunger. The syringes loaded with the polymers and formulations of broad molecular weight and polydispersity of the old method where inconsistent, only two syringes were able to release the polymer or the formulation using common force on plunger while three failed injectability and required extra force for allowing the formulation to pass through the needles with one syringe of the polymer and one of the formulation did not allow any release at room temperature.

In vitro release was determined by placing one gram of the formulation into plastic containers (vial cover) covered with a plastic net and settled at the bottom of an 800 ml glass container. The release study was done in a medium of buffer acetate (pH=4.5) (consisting of NaCl: 3.41 gram/liter, Acetic acid: 3.33 ml/liter, Sodium acetate: 3.41 g/liter) at 37° C. at 10 rpm shaking. For comparison, the release was also determined in phosphate buffer pH7.4 at 37° C.

GM analysis in the release media was determined by UV. Calibration curve was prepared in a concentration range of 1-16 μg/ml where GM was reacted with 200 μl of 0.1 mg/ml fluorescamine solution in acetone, sample volume was made up to 2 ml using borate buffer pH-7, incubated for 15 min at room temperature and analyzed by spectro-fluorimeter at excitation wavelength 390 nm and emission wavelength 460 nm. The amount released was calculated based on the calibration curve that was prepared at the same day of the determination of gentamicin from the release solutions.

The gentamicin content in the remaining formulation samples was determined by adding 20 ml of chloroform in the formulation and a vortex was done for two minutes. The mixture was kept in 37° C. for four hours and then 20 ml of acidic DDW (pH=2) was added and then mixing was done with vortex. In order to get two separated phases, centrifugation at 4000 RPM for 10 minutes was used. The upper (DDW) phase of the sample was taken in order to analyze the amount of GM remaining in the polymer after release. Gentamycin concentration was determined by spectrofluorometer using fluoresceine. A recovery of about 80% of the gentamicin content could be recovered from the polymer formulations.

Gentamicin was constantly released for 28 days in the pH 4.5 media. The release media solutions were replaced every week with fresh buffer solution. After 28 days, the gentamicin content in the remaining formulation was determined. Formulations prepared with the polymers of this invention, showed almost linear release profile for the entire 28 days with a narrow standard deviation of between 1 and 5% of each data point. about 60% of GM being released. Recovery of gentamicin from the remaining polymer formulation was about 20% of the original gentamicin content, no full recovery was obtained. The release in neutral pH, phosphate buffer pH7.4 was constant for the first week where about 20% of GM was released and later only little was released due to probably salt formation between GM and the acidic degradation product oligomers that are less water soluble.

The release of gentamicin from the polymer formulations of the old method was constant for the 28 days but the standard deviation was between 5 and 20% of the amount released at each time point. The recovery of gentamicin from these polymer formulations was between 10 and 25% of the original content.

### Example 5: A Comparison of the Release Rate of Gentamicin from Irradiated and Non-Irradiated Formulations

Gentamicin formulations in P(SA:RA)(30:70) with a loading of 20% (w/w)) and a formulation loaded in glass syringes, after irradiation with 2.5 Mrad dose was used in this study. Formulation, 200 mg, were loaded in plastic caps with a covered area of about 1.76 cm² and immersed in a 100 ml phosphate buffered saline pH 7.4 consisting of NaCl 8 g/L, KCl 0.2 g/L, Na₂HPO₄ 12H₂O 2.9 g/L, KH₂PO₄ 0.24 g/L. The vials were placed on an orbital shaker 30 RPM in an oven at 37° C. Samples of 2 ml were taken after 1, 8, 24, 48, 72 and 168 hours. After 24, 72, 168 hours the medium was replaced by fresh buffer.

Both, the irradiated and non-irradiated samples were of similar viscosity with no change in molecular weight or appearance. Both released the loaded GM in a constant manner for the duration of the study. About 50% of the GM was released with about 15-20% of the drug recovered from the remaining formulation. FT-IR spectra of the formulation before the release study indicates that there are ester and anhydride bonds, and after 168 hours of release only little anhydride bonds are in the polymer but high ester and carboxylic acid peaks. Both irradiated and non-irradiated formulations show similar FTIR spectra.

### Example 6: Toxicity of Gentamicin Sulfate Loaded PSARA 30:70

The potential toxicity test items: 10% and 20% loaded gentamicin sulfate in PSARA 30:70 paste of this invention and the blank polymer carrier. These formulations were injected subcutaneously to Sprague Dawley rats to determine MTD.

The study was performed as follows: 6 groups of rats, 6 rats in each group, 3 male and 3 females. Three groups were injected with 0.2 ml of either the blank polymer, 10% loaded gentamicin and 20% gentamicin. The other three groups were same but the injection dose was 0.4 ml. The animals were followed for 14 days and sacrificed. At the end of the study, general necropsy was performed and the skin of injection sites was submitted for histopathology.

Dosing materials were provided ready for use. Each Dosing Material was thawed on the day of dosing and transferred to an injection syringe pre-fitted with a 19G thin wall needle via the plunger end directly from the syringes. No mortality occurred in any of the animals treated or placebo and saline control throughout the 14-day study period.

No noticeable treatment related systemic reactions were observed in any of the test items. A local reaction at injection site in the form of a subcutaneous bulge was observed in all animals assigned to the study from the day of dosing and until the scheduled sacrifice at 14 days post dosing. No local reactions were noted in any of the saline controlled animals throughout the entire 14-day observation period.

All animals made expected body weight change at the end of the 14-day period.

At necropsy, all tested animals displayed capsule-like mass, usually filled with firm substance. Saline control treated animals showed no gross pathological findings.

Histopathological evaluation revealed comparable tissue reaction at injections site in terms of size and nature among all tested groups. The reaction was composed of central cavities region, surrounded by layer of granulomatous inflammation, and more externally by fibrotic layer. The empty cavities (mostly of grade 3-moderate) are suggested to reflect the washed out injected material. The granulomatous inflammation layer (mostly of grade 2-mild) was composed of mixture of mononuclear cells, macrophages and multinucleated giant cells. The fibrotic layer (mostly of grade 3-moderate) was composed of fibroblasts embedded in collagen. The granulomatous reaction was expected to be seen following injection of a foreign material which is progressively absorbed. The presence of gentamicin was not associated with any increase in the nature, grade and extend of inflammation, comparing to the group injected with the placebo for gentamicin implant system. No lesions were seen in the group of animals injected with saline.

### Example 7: Efficacy Assessment of p(RA-SA)-Containing 20% w/w Gentamicin

The effectiveness of p(RA-SA)-containing 20% w/w gentamicin to eliminate the bacteria and reduce the negative consequences of osteomyelitis on bone healing was tested. Gentamicin, which is the antibiotic released from p(RA-SA)-containing 20% w/w gentamicin, is an aminoglycoside, that is commonly used both in humans and in animal models to treat or prevent osteomyelitis, due to thermostability and wide antibacterial spectrum. An osteotomy model for S. aureus artificial contaminated open fracture was established. This model provides improved reproducibility among multiple animals in comparison to induction of traumatic fracture. The radius bone was used due to low mechanical burden. In addition, the surrounding muscles and the parallel ulna bone contribute to the fracture mechanical support without additional fixators. The criteria for effective treatment was the S. aureus count in the bone suspension of bone isolated from the site of inoculation and treatment. Three groups of animals, 6 in each group, were used, one without treatment, one treated with the polymer only and one with p(RA-SA)-containing 20% w/w gentamicin. The experiment was lasted for 28 days where in the last day, the animals were sacrificed and the bones at the site of the S. aureus inoculation were isolated, crashed into a suspension in sterilized buffer solution and tested for bacterial content.

All 6 samples in group 1 (contaminated, no treatment) were positive for S. aureus and propagated similarly to the samples in group 2 (p(RA-SA) only). S. aureus counts of >1000 CFU/ml were detected. In group 3 (contaminated, local treatment with p(RA-SA)-containing 20% w/w gentamicin), all 6 animals were negative for S. aureus. Thus, the local treatment with p(RA-SA)-containing 20% w/w gentamicin was successful in complete eradication of the S. aureus bacteria that was used to induce the contamination. Microbial examinations therefore revealed that administration of p(RA-SA)-containing 20% w/w gentamicin led to excellent culture assessments, with no growth of S. aureus in any of the samples.

No mortality occurred in any of the animals throughout the 28 days observation period. Avoidance of using the operated limb was observed during the first few days post-fracture induction. During the third and fourth week, this behavior was seen in 1 animal from group 3 and in all animals of groups 1 and 2. Swelling of the treated bone was seen in most animals of the control groups 1 and 2. Decrease in all animals' body weight was noted during the first week post-fracture induction, however, by Day 9 all animals regained their initial body weight and demonstrated expected growth pattern until the end of the observation period.

### Example 8: In Vitro Release of Antimicrobial Agents

The following antimicrobial agents were incorporated in p(RA-SA)70:30 of this invention: tobramycin, erythromycin, vancomycin, ciprofloxacin, chlorhexidine, amphotericin B, cefuroxime, ketoconazole, levofloxacin, clindamycin, azithromycin and acyclovir. All agents were dry powders that were hand mixed in the polymer paste at room temperature at concentrations of 5, 10 and 20% w/w and loaded in 2 ml glass syringes and the injectability through a 19G needle, release profile and stability over three months at room temperature was determined. For all agents, uniform opaque pastes were obtained of different viscosities. As the drug content increases from 5 to 20%, an increase in viscosity of the resulted paste was noted. All formulations showed good injectability and did not show any change in appearance, drug content and viscosity during the 3 months of storage. The in vitro release was evaluated for one week where the release media was adjusted to the agent released and the concentration in the release media was determined mainly by UV. All agents showed a constant release with 5 to 50% of the loaded agent being released during the release study. In general, the higher the drug content, the faster is the observed release. Amphotericin B, a highly water insoluble agent, released only minimal about to phosphate buffer solution, however, when adding 1% Span 80 to the release media, faster release was obtained.

### Example 9: In Vivo Gentamicin Release and Polymer Elimination

The objective of the study was to determine gentamicin release to injection site and to the blood, as well as polymer elimination from the site of injection. Animals were clinically observed for up to 8 weeks post-dosing. At the end of the respective observation period and other pre-determined time points, blood-plasma, injection site and surrounding areas were collected from each animal and transferred for gentamicin analysis and histopathology.

Blood and muscle samples at the injection site, at various time points following a single intramuscular injection of p(RA-SA)-containing 20% w/w gentamicin to male NZW rabbits towards assessment of the degree of gentamicin local release from the carrier polymer.

Pre-filled syringes containing 0.5 ml formulation per syringe were used. The formulation was administered by the intramuscular route, at 0.2 ml/animal to anesthetized animals. The formulation was administered by a single slow injection to the right mid paravertebral muscle (˜2.5-5 cm from the spinal cord and approximately 1 cm in depth). All animals either maintained their weight or gained weight with no clinical signs of illness. All blood-plasma and muscle samples were collected on schedule.

Gentamicin was found in blood only during the first 24 hours post injection at very low levels and below detection levels thereafter. Gentamicin concentrations in muscle at the injection site of 4 mm diameter of the injection point, showed very high concentrations of >100 microgram per gram tissue for the first 3 weeks and reduction to ˜5-10 microgram per gram tissue in the weeks after. Gentamicin concentration reduced significantly in tissues that are at a distance of 10 and 15 mm from the injection site. Histopathology of the injection site at the end of the 8 weeks, indicated only minor signs of inflammation with only traces of the polymer formulation in the site of injection.

A similar study was conducted in rats where rats were injected intramuscularly with 0.05 ml of the polymer-gentamicin formulation and the gentamicin blood levels and at the muscle at the injection site were determined. Concentrations of >1000 microgram per gram tissue were found at the injection site during the first 24 hours. The drug concentrations at the injection site reduced exponentially with time where after 3 weeks the concentration was ˜100 microgram per gram tissue and ˜5 after 8 weeks. Gentamicin blood levels of 1-8 microgram per ml were found at 2 hours post injection and below detection level after 6 hours. No signs of toxicity were observed at all times during the study. Histopathology of the injection site after 8 weeks did show almost complete healing which almost no signs of the injected material. These studies indicate controlled release of gentamicin at the injection site with no systemic distribution after 6 hours post injection.

### Example 10: Acetic Acid/Anhydride Content in Polyanhydrides Prepared with Different Amounts of Acetic Anhydride

The concentration of acetic anhydride and acetic acid content in poly(RA:SA)70:30 synthesized with excess 1:5 acetic anhydride to oligomer carboxylic acid content or with 0.8:1 molar ratio was determined by GCMS. The polymers were dissolved in dichloromethane and immediately injected to GCMS for acetic acid/anhydride determination. The polymers prepared with excess acetic anhydride showed traces of acetic acid/anhydride in the range of 10-100 ppm while the polymers prepared with mole equivalent or less of acetic anhydride did not show any acetic acid/anhydride in the polymer samples. Polymers containing acetic acid or anhydride may react with the incorporated drug to form new molecular entities or being released and reduce the pH in the surrounding tissue which may affect the tissue.

### Example 11: Shelf-Life Stability of Poly(RA:SA)70:30

Glass syringes loaded with 0.5 g poly(RA:SA)70:30 paste, packed in aluminum foil envelopes under vacuum were places in cabinets of the following temperatures: −20, 4-8 and 25° C. and the molecular weight was determined by GPC, viscosity by viscometer and anhydride bonds content by FTIR. No change in molecular weight, viscosity and FTIR spectra was observed.

The polymers of this invention are stable at room temperature for months, possess batch to batch high reproducibility with narrow polydispersity, does not contain traces or acetic acid or anhydride, incorporation of active agent is at room temperature with gentle mixing, various powdery agents can be formulated in the polymers and the obtained pasty formulation is injectable, drug loading of 20 or even 30% is possible, possess a highly reproducible batch to batch release profile of incorporated agents, high reproducibility in in vitro degradation profile. The polymers of this invention are highly biocompatible, degrade to natural fatty acids that are easily eliminated from the body. The polymer carrier confines the release of incorporated drugs to the site of injection with minimal systemic distribution of the incorporated agent. Moreover, two or more active agents may be incorporated and released from the polymer for controlled release applications. The polymers of this invention are not affected by irradiation sterilization.

1-48. (canceled)

49. An antimicrobial formulation comprising at least one antibiotic agent and a carrier in a form of a polyanhydride composed of sebacic acid (SA) and ricinoleic acid (RA), the carrier having a Mw/Mn value between 1 and 2.5.

50. The formulation according to claim 49, wherein the carrier is a polyanhydride of the formula —(SA-RA)n-, wherein n is an integer between 10 and 100.

51. The formulation according to claim 49, wherein the polyanhydride is prepared by: a. melt condensation of SA and RA to form dicarboxylic acid oligomers; b. oligomer activation with acetic anhydride; c. melt polycondensation to form a polyanhydride, wherein the preparation does not comprise use of poly sebacic acid.

52. The formulation according to claim 51, wherein the oligomer activation is in the presence of a mole equivalent or less of acetic anhydride per carboxylic acid groups, in the absence of a solvent.

53. The formulation according to claim 49, in a form of an implantable formulation or device or an injectable formulation.

54. The formulation according to claim 49, wherein the antibiotic agent is effective against a bacterium or a parasite.

55. The formulation according to claim 54, wherein the bacterium is selected amongst cocci bacteria, bacillus bacteria, rickettsia bacteria, and mycoplasma bacteria.

56. The formulation according to claim 54, wherein the bacterium is selected amongst Gram-positive and Gram-negative bacteria.

57. The formulation according to claim 49, wherein the antibiotic agent is selected to treat or prevent an infection caused by Gram-positive bacteria.

58. A method for treating or delaying or preventing progression of an infection or a disease mediated or caused by a bacterium, the method comprising administering an effective amount of an antibiotic agent in a formulation comprising a carrier in a form of a polyanhydride of the formula —(SA-RA)n-, wherein SA is sebacic acid and RA is ricinoleic acid, and wherein n is an integer between 10 and 100, the carrier having a Mw/Mn value between 1 and 2.5 or 1 and 2.

59. The method according to claim 58, wherein the polyanhydride is prepared by melt condensation of SA and RA.

60. The formulation according to claim 59, wherein the melt condensation is in the presence of a mole equivalent or less of acetic anhydride per carboxylic acid group, in the absence of a solvent, and wherein the preparation does not comprise use of poly sebacic acid.

61. A method for treating or delaying or preventing progression of an infection, the method comprising administering an effective amount of an antibiotic agent in a formulation comprising a carrier prepared by melt condensation of SA and RA.

62. The method according to claim 61, wherein the carrier is in a form of a polyanhydride of the formula —(SA-RA)n-, wherein SA is sebacic acid and RA is ricinoleic acid, and wherein n is an integer between 10 and 100, the carrier having a Mw/Mn value between 1 and 2.5 or 1 and 2.

63. The method according to claim 61, wherein the formulation is administered by injection.

64. The method according to claim 61, wherein the formulation is administered topically or systemically.

65. The method according to claim 61, wherein the formulations is administrated by an administration mode selected from transmucosal, transnasal, intestinal, parenteral, intramuscular, subcutaneous, intramedullary injections, intrathecal, direct intraventricular, intravenous, intraperitoneal, intranasal, or intraocular injections.

66. The method according to claim 65, wherein the formulation is administered by injection.

67. The method according to claim 61, wherein the formulation is administered by implanting same into a tissue or an organ.

68. A kit comprising an antibiotic drug and a carrier in a form of a polyanhydride of the formula —(SA-RA)n-, wherein SA is sebacic acid and RA is ricinoleic acid, and wherein n is an integer between 10 and 100, the carrier having a Mw/Mn value between 1 and 2.5 or 1 and 2; and instructions of use.
